(19) United States
(12) Patent Application Publication
(10) Pub. No.: US 2015/0359757 A1
(43) Pub. Date: Dec. 17, 2015

(54) A METHOD FOR IMPROVING THE WELLBEING OF AN ANIMAL USING A COMPOSITION INCLUDING VITAMIN K

(71) Applicants: Hubertus Leonardus REGTOP, Mittagong, NSW (AU); John Ray BIFFIN, High Range, New South Wales (AU)

(72) Inventors: Hubertus Leonardus Regtop, Mittagong, NSW (AU); John Ray Biffin, High Range, NSW (AU)

(21) Appl. No.: 14/762,722

(22) PCT Filed: Jan. 21, 2014
(86) PCT No.: PCT/AU2014/000035

§ 371 Date: Jul. 22, 2015

(30) Foreign Application Priority Data
Jan. 22, 2013 (AU) 2013900201
May 15, 2013 (AU) 2013901713

(51) Int. Cl. A61K 31/122; A61K 47/48; A61K 31/015; A61K 31/59; A61K 47/36; A61K 9/50; A61K 47/02

(52) U.S. Cl. CPC A61K 31/122; A61K 9/50; A61K 47/48969; A61K 47/02; A61K 31/59; A61K 47/36; A61K 31/015

(57) ABSTRACT

There is disclosed herein a method of improving the wellbeing, as herein defined, of an animal comprising administering to an animal an effective amount of a composition containing: vitamin K1, vitamin K2 or a mixture of vitamin K1 and vitamin K2, together with a physiologically acceptable carrier, excipient and/or diluent.

### TECHNICAL FIELD

The present invention relates in one embodiment to a method for improving the wellbeing of an animal such as pigs, turkey, ducks and/or chickens by administering a bioavailable (water soluble) and UV-stable composition containing vitamin K1, vitamin K2 or a mixture of vitamin K1 and K2 with or without vitamin D.

### BACKGROUND OF THE INVENTION

The information provided herein and references cited are provided solely to assist the understanding of the reader, and do not constitute an admission that any of the references or information is prior art to the present invention.

Factory farming or intensive farming is a process of raising livestock in confinement at high stocking densities. At least 75% of the world's production of meat, eggs and milk are produced by intensive farming. Confinement at high stock density is one part of a systematic worldwide effort to produce the highest output and the lowest cost by relying on economies of scale, modem machinery, biotechnology and global trade.

Intensive farming however has a number of disadvantages.

Intensive farming of animals causes stress, a result of the animal being contained in the confined space. Such stress may result in animals attacking one another and cannibalism. In an effort to prevent injury, tails and teeth of the animal may be removed. De-beaking of poultry is one recognised method used to deter fighting and cannibalism.

Confinement and overcrowding of animals also prevents the animals from exercising. Lack of exercise weakens bones and muscles as well as causing a dramatic increase in bodyweight, a function also of a high density feed intake. The skeletal structure of the animal is not able to support this increase in bodyweight.

Animals crowded together in a closed, warm and dusty environment are highly prone to the transmission of contagious and infectious diseases. Colibacilliosis is one economically undesirable disease which has been implicated in a variety of conditions in poultry and other animals (e.g., pigs) and causes diseases such as colisepticaemia, coligranuloma, air sac sacculitis, peritonitis, pericarditis, omphalitis and oophoritis (accounting for about 5-50% mortality). Selecting animals for faster growth rates and higher meat yields leave an animal's immune system less able to cope with these infections and there is a higher degree of genetic uniformity in the population making the spread of disease more likely.

In order to overcome these disadvantages, the use of antibiotics and growth promoters are used extensively and their use has risen with the intensification of livestock farming. Infectious agents reduce the yield of farmed food animals and, to control this, the administration of sub-therapeutic antibiotics and antimicrobial agents is effective. Antimicrobial therapy helps in reducing the incidence of mortality associated with avian colibacillosis. For example, penicillins (e.g. phenoxymethyl penicillin, amoxycillin) in drinking water or bacitracin in feed (e.g. 100 ppm) may be used for chickens and turkeys, however treatment of ducks is not very successful. Neomycin and erythromycin are typically used in the USA with water medication for 3-5 days and in-feed medication for 5-7 days depending on the severity.

According to the National Office of Animal Health (NOAH, 2001), antibiotic growth promoters are used to “help growing animals digest their food more efficiently, get maximum benefit from it and allow them to develop into strong and healthy individuals”. Although the mechanism underpinning their action is unclear, it is believed that the antibiotics suppress sensitive populations of bacteria in the intestines. It has been estimated that as much as 6 per cent of the net energy in the pig diet could be lost due to microbial fermentation in the intestine: Jensen B, 1998 Journal of Animal and Feed Sciences, 7, 45. If the microbial population could be better controlled, it is possible that the lost energy could be diverted to growth.

Thomke, S et. al, (1998), Ann. Zootech, 47, 245 hypothesize that cytokines released during the immune response may also stimulate the release of catabolic hormones, which reduce muscle mass. Therefore a reduction in gastrointestinal infections would result in the subsequent increase in muscle weight.

Whatever the mechanism of action, the result of the use of growth promoters is an improvement in daily growth rates between 1 and 10 per cent, resulting in meat of a better quality, with less fat and increased protein content.

There can be no doubt that growth promoters are effective. Prescott. J, Antimicrobial Therapy in Veterinary Medicine, 2nd Edition, Iowa State University Press showed that the effects of growth promoters were much more noticeable in sick animals and those housed in cramped, unhygienic conditions.

There has been however developing controversy surrounding the use of antibiotics as growth promoters for food animals. Use of any antibiotic is associated with the selection of resistance in pathogenic bacteria and it has been argued that the use of antibiotic growth-promoters imposes a selection pressure for bacteria that are resistant to antibiotics that may be used in clinical or veterinary practice, thus compromising the continued use of antimicrobial chemotherapy. Also there is the, issue of antibiotic resistance transference to humans.

Problems caused by antibiotic use are largely those of developed rather than developing countries.

It would be desirable to provide a method for improving the wellbeing of animals without the need for use of such antibiotics or growth promoters.

### SUMMARY OF THE INVENTION

According to a first aspect of the present invention, there is provided a. method of improving the wellbeing of an animal comprising administering to an animal an effective amount of a composition containing:
vitamin K1, vitamin K2 or a mixture of vitamin K1 and vitamin K2, together with a physiologically acceptable carrier, emulsifier, excipient and/or diluent.

### DEFINITIONS

The following are some definitions that may be helpful in understanding the description of the present invention. These are intended as general definitions and should in no way limit the scope of the present invention to those terms alone, but are put forth for a better understanding of the following description.

Unless the context requires otherwise or specifically stated to the contrary, integers, steps or elements of the invention recited herein as singular integers, steps or elements clearly encompass both singular and plural forms of the recited integers, steps or elements.

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

By “improving wellbeing” means one or more of increased vitality, reduced infection rates, minimised aggressive behaviour, even growth, weight gain, increased mobility, increased egg laying, reduction of misshapen eggs, decreased egg breakage, increased metabolic calcium deposition in eggs, improved plumage and increased meat quality. Improving wellbeing does not include increasing bone density, maintaining bone density, inhibiting loss of bone density, reducing osteochondral defects or increasing plasma level of vitamin K beyond that achievable by diet.

“Vitamin KQ” in this application is a proprietary combination of K1 and/or K2 optionally together with vitamin D which has been made UV-stable and bio-available (water soluble). This composition typically contains a UV absorber and is suitably an emulsion.

### DETAILED DESCRIPTION OF PREFERRED EMBODIMENTS OF THE INVENTION

The major clinical sign of vitamin K deficiency noticed in all animal species is the impairment of blood coagulation. Clinical signs include, but are not limited to, increased clotting time and hemorrhage. Vitamin K deficiency can also lead to impaired bone mineralization due to inadequate carboxylation of osteocalcin, a protein involved in bone mineralization.

Deficiencies may result from inadequate vitamin K in the diet, disruption of microbial synthesis within the gut, prevention of coproghagia, inadequate absorption from the intestine, ingestion of vitamin K antagonists (substances that counteract the effect of vitamin K), or the inability of the liver to utilize available vitamin K or organs unable to synthesise various forms of vitamin K.

Green leafy vegetables are regarded as a good source of vitamin K Vitamin K is also found in liver, meat, milk, and egg yolk.

Vitamin K can exist in three forms, two of them are naturally occurring and one a synthetic analogue:
Vitamin K1, also known as phytonadione, phylloquinone, or phytomenadione is the form of vitamin K that occurs naturally in live green plants. It is extremely UV-labile and is destroyed in a cut plant in hours. It is also destroyed in cut plants by fluorescent light;
Vitamin K2, or menaquinone, also naturally occurring, is the fat soluble form of vitamin K synthesized by the bacteria or bacteria in the intestinal tract. Also found in milk and fermented products as bacteria synthesize a range of related forms of this vitamin. These vitamin K analogues are collectively known as K2; and
Vitamin K3, also known as menadione, is the synthetic, water soluble analogue of vitamin K that can be converted to K2 in the intestine but pharmacologically has little or no activity as there is poor absorbance in the colon as measured by prothrombin activity.

Different sources of vitamin K, including those that are listed in the Association of American Feed Control Officials' Official Publication as accepted for use in animal feed, are broadly denoted as vitamin active substances.

Vitamin K1 is not used by most feed companies as it is expensive and unstable, instead, water soluble menadione (vitamin K3) salts are used to provide vitamin K activity in feeds. Because of instability, menadione is not used in feed as a pure vitamin and hence is formulated as water soluble salts which includes menadione sodium bisulphite (MSB), menadione dimethyl-pyrimidol bisulphite (MPB) and menadione nicotinamide bisulphite (MNB).

In the United States, menadione supplements are banned by the U.S. Food and Drug Administration (FDA) because of their potential toxicity in human use.

Low dose menadione is still used as an inexpensive micronutrient for livestock in many countries. Forms of menadione are also included in some pet foods in developed countries as a source of vitamin K. These doses have yielded no reported cases of toxicity from menadione in livestock or pets.

The US Food and Drug Administration recommends that this substance menadione can be added to chicken and turkey feeds at a level not to exceed 2-4 g per ton of complete feed, and to growing and finishing swine feeds at a level not to exceed 10 g per ton of complete feed.

Stability of menadione water soluble salts is excellent in multivitamin premixes unless trace minerals are present: Fry, T. M “Vitamin compatability in custom premixes” Vitamin Nutrition Update—Seminar Series 2, p 70, RCD 5483/1078, Hoffman La Roche. Stability in complete feed formulation of multivitamins, choline chloride and trace mineral premix indicates less stability. Tests carried out at room temperature for 4 months showed MSB retained 33%, MPB 57% and MNB 83%: Huyghebaert, A. 1991 “Stability of vitamin K in a mineral premix,” World Poultry, 7:71.

Substances with vitamin K activity are often added to animal diets to ensure that animals do not develop haemorrhagic disease. Even though vegetable sources contain fairly high amounts of vitamin K1, until recently little was known about the actual bioavailability of the vitamin from these sources.

The bioavailability of phylloquinone from vegetable sources in humans is anything from 4.7-15%: Gijsbers B. et al., 1996, British Journal of Nutrition, 76, 223; Novotny et al., 2010, British Journal of Nutrition, 104(6), 852-862.

Vitamin K tolerances in animals based on the limited amount of available information show that vitamin K1 does not result in any toxicity even when 25 gm/kg of phylloquinone, the natural form of vitamin K, is consumed. It is also noted however that menadione, the synthetic vitamin K3 reaches toxic levels at the dose of 0.5 mg/kg (at least 50× more toxic than vitamin K1): Molitor H. at al., 1940, Proceedings of the Society for Experimental Biology and Medicine, 43, 125.

Suttie J. W, 1980, CRC Critical Reviews in Biochemistry, 191 reviewed the various forms of vitamin K's on their capacity for carboxylation. Friedman P. et aL, 1980, Biochimica et Biophysica Acta, 616, 362 studied the carboxylation of endogenous precursors and found that the relative activity of K1 was 4 times the activity of menadione Vitamin K3. In fact menadione (Vitamin K3) has very little effect on prothrombin synthesis (approximately 100 times lower in animals): Jones J. at al., 1976, Biochemical and Biophysical Research Communication, 72, 589.

A dose of 2.5 umole of menadione/kg 430 ugK3/Kg bwt. in chickens causes inhibition of calcium transfer from luman-blood within 30 min and lasting 6-10 hrs: A. Marchionetti et al, 2003, Journal of Nutritional Biochemistry, 14, 466; Biochemica et Biophysica Acta, 2008, 1780, 101.

It was previously thought that vitamin K's main role was in the carboxylation of four coagulation factors F11, FX, FV11, F1X involved in the clotting process and hence the prevention of haemorrhage in animals but recently three anticoagulant proteins which are also carboxylated have been discovered Protein C, S and Z.

There are sixteen proteins now identified which are carboxylated and have various biochemical and physiological roles in humans and animals: McCann. J. et al., 2009, American Journal of Clinical Nutrition, 90, 889.

Dietary Vitamin K is usually inadequate to maximize carboxylation of osteocalcin, the amount required to maximize osteocalcin is now known in humans. The third nutrition and health survey has set 120 and 90 ug/day for men and women respectively, The NHMRC RDI in humans of Vitamin K1 is 100 ug to maximize prothrombin activity, but 1000 ug has been found to maximize osteocalcin: Binkley N. C et al., 2002, American Journal of Clinical Nutrition, 76, 1055. The recommendation in Japan is 45-90 mg of MK4 and in Europe 1000 ug of MK7.

It has been shown that horses require supplementation of Vitamin K1 as grasses are not sufficient to maximize carboxylation of osteocalcin, because of poor bioavailability and hence need supplementation: Biffin, et al., 2008, Proc. Aust. Equine Sc. Symp., vol. 2, p 36; Biffin, et al., 2010, Proc. Aust. Equine Sc. Symp., vol. 3, p 18-19.

During the course of bacterial infection, innate immune cells such as neutrophils and macrophages sense bacteria and bacterial products such as lipopolysaccharides or peptidoglycans etc., these compounds are recognised on receptors on innate immune cells including toll-like receptors. Once recognised, these cells release proinflammatory cytokines to recruit neutophils and additional macrophages. These neutrophils and macrophages ingest and kill the bacteria.

Many other receptors are implicated in the recognition and phagocytosis of bacteria.

Toll like receptors (TLR's) are crucial triggers of innate immunity through the recognition of pathogenic molecular markers. To date 11 TLR's have been found in humans and 13 in mice.

The TLR activation pathway induces the production of numerous pro-inflammatory cytokines including interleukins, TNF and NF kappa B. These cytokines are involved with the hosts defence of pathogens through the regulation of immune responses or direct killing of invading pathogens.

Although TLR-mediated inflammation is essential for host defence against pathogens, TLR signalling must be tightly controlled because unrestrained TLR activation generates chronic inflammatory and can result in chronic inflammation disorders.

Several TLR signalling suppressors have been described in immune cells, and recent studies have revealed that Tyro3, Axl and Mer (TAM) receptors play a pivotal role in negatively regulating innate immunity via the inhibition of the TLR mediated inflammatory response and the promotion of phagocytic clearance of apoptotic cells. Vitamin K suppresses lipopolysaccharide induced inflammation in animals: Ohsaki. Y. et al., 2006, Bioscience Biotechnology Biochemistry, 70, 926.

Vitamin K activates GAS6 which is a ligand for Tyrosine Kinase Axl. Activated GAS6 binds to phosphatidylserine on membranes while GAS6 which is not activated does not bind: Hasanbasic. I. of al., 2005, Journal of Thrombosis and Haemostasis, 3, 2790.

GAS6 has been proposed as a broad regulator of the innate immune response. GAS6 synthesis is therefore likely to be a regulatory mechanism during systemic inflammation: Hurtado B of al., 2010, Critical Care, 14, 1003.

The present inventors have used Vitamin KQ to replace the use of growth promoters and antibiotics and improve wellbeing of an animal including improving quality of meat, quality of eggs and providing calmer and non-aggressive animals.

The present inventors have discovered that by increasing the bioavailability of vitamin K1/K2 and hence increasing the levels of vitamin K1/K2 and not Vitamin K3 in the amounts recommended by the USA Food and Drug administration in food or water for chickens, ducks, turkeys and pigs improves the wellbeing of these animals. Suitable formulations for use are such as described in Australian Patent Application 2008902795 and US Patent Application 2011/0124610 A1, incorporated herein by reference.

Undesirable factors associated with intensive overcrowding farming procedures include:
1) Increased infection rates and the use of antibiotics and growth promoters;
2) Aggression and pecking due overcrowding and stress leading to de-beaking, tail removal in pigs and removal of teeth;
3) Bone breakage, weak legs and reduced mobility due to less structural strength in bones because of large bodyweight and less calcium deposition in bone
4) Decreased egg laying due to the age of the chicken and misshapen eggs in poultry due to stress and overcrowding;
5) Increased breakage in eggs due to stress and metabolic decreased calcium deposition; and
6) Reduced meat quality due to stress and overcrowding and deficient nutrient intake.

The present inventors have found that by administering Vitamin KQ (a bioavailable and UV-stable combination of vitamin K1/K2 with or without vitamin D) and trademarked “Quinaquanone” results in improvement of wellbeing and reduction of these undesirable factors.

Having regard to the above, the present invention provides a method of improving the wellbeing, as herein defined, of an animal comprising administering to an animal an effective and stable amount of a composition containing:
vitamin K1, vitamin K2 or a mixture of vitamin K1 and vitamin K2, together with a physiologically acceptable carrier, emulsifier, excipient and/or diluent.

In one embodiment the composition comprises a UV stabilizer/absorber.

The animal can be a porcine or avian. In one embodiment the animal is a pig, chicken, turkey or duck.

In one embodiment, the composition comprises vitamin KQ (a bioavailable and UV stable combination of vitamin K1/K2 with or without vitamin D) and trademarked “Quinaquanone”.

The composition can be administered orally, parenterally, intramuscularly, by injection, or intravenously.

The composition can also be in the form of a slow-release composition.

A suitable composition according to one embodiment comprises:
vitamin K1, vitamin K2 or a mixture of vitamin K1 and vitamin K2; and a UV absorber.

The composition may further comprise an emulsifier and/or thickener.

The composition may further comprise vitamin D such as vitamin D3.

In one embodiment a composition for use in the invention is a stable and water soluble composition comprising:
vitamin K1, vitamin K2 or a mixture of vitamin K1 and vitamin K2;
beta-carotene; and
an emulsifier and/or thickening agent and/or a water miscible or soluble powder.

The composition may be in the form of a liquid or a paste.

A composition according to another embodiment for use in the invention is a stable powder composition comprising:
vitamin K1, vitamin K2 or a mixture of vitamin K1 and vitamin K2; and
beta-carotene;
an emulsifier and/or thickening agent;
encapsulated into a modified starch and/or a zeolite.

A composition according to another embodiment for use in the invention is a stable powder composition comprising:
vitamin K1, vitamin K2 or a mixture of vitamin K1 and vitamin K2;
beta-carotene;
Vitamin D;
an emulsifier;
encapsulated into a modified starch and/or a zeolite.

In one embodiment, the vitamin K from the powder compositions is soluble when added to water.

Vitamin K1, vitamin K2 or combination of vitamin K1 and K2 may be present in an amount of from 0.01 wt % up to 99.9 wt % of the composition, for example a range of 1 to 99 wt %, or a range of 1 wt % to 10 wt %, 11 wt % to 20 wt %, 21 wt % to 30 wt %, 31 wt % to 40 wt %, 41 wt % to 50 wt %, 51 wt % to 60 wt %, 61 wt % to 70 wt %, 71 wt % to 80 wt %, 81 wt % to 90 wt % or 91 wt % to 99%, for example 95 wt %, 90 wt %, 80 wt %, 70 wt %, 60 wt %, 50 wt %, 40 wt %, 30 wt %, 20 wt %, 10 wt %, 5 wt %, 1 wt %, 0.5 wt %, 0.2 wt %, 0.15 wt % or 0.1 wt %. In one embodiment a UV absorber is present in an amount of 1/10th or less of the concentration of vitamin K1 and/or vitamin K2. In one embodiment vitamin D3 is present in an amount, of 3 times the concentration of vitamin K1 and/or vitamin K2.

The composition may include a UV absorber. The UV absorber may be a zinc oxide, lycopene, lutein or beta-carotene. The composition may also include vitamin D3. The UV absorber and vitamin D3 may be present in respective amounts up to 10 wt %, for example a range of 0.01 to 10 wt % of 1 to 9 wt % or for example, of 1 to 5 wt %), or for example 5.0 wt %, 3.0 wt %, 2.0 wt %, 1.5 wt %,1.0 wt %, 0.5 wt %, 0.25 wt %, 0.2 wt % or 0.15 wt %.

In another embodiment the UV absorber and vitamin D3 are present in a range of 0.01 wt % up to 99.9 wt % of the composition, for example a range of 1 to 99 wt %, or irange of 1 wt % to 10 wt %, 11 wt % to 20 wt %, 21 wt % to 30 wt %, 31 wt % to 40 wt %, 41 wt % to 50 wt %, 51 wt % to 60 wt %, 61 wt % to 70 wt %, 71 wt % to 80 wt %, 81 wt % to 90 wt % or 91 wt % to 99 wt %, for example 95 wt %, 90 wt %, 80 wt %, 70 wt %, 60 wt %, 50 wt %, 40 wt %, 30 wt %, 20 wt %, 10 wt %, 5 wt %, 1 wt %, 0.5 wt %, 0.2 wt %, 0.15 wt % or 0.1 wt %.

The composition may be in the form of a liquid, paste or powder. The composition may be in the form of a beverage, soup, concentrate, suspension, emulsion, pill, granules, tablets, capsules, suppository, controlled-release composition, cream, ointment, or salve.

The composition may be administered daily or twice daily or more or less frequently in a single dose or in ,several doses.

The composition may include a weight ratio of K1:K2 of 1:9.9 to 9.9:1, for example from 1:9 to 9:1, 2:8 to 8:2, 3:7 to 7:3, 4:6 to 6:4 or 5:5.

In one embodiment, there is provided a stable and water soluble powder composition for use in the invention comprising:
vitamin K1, vitamin K2 or a mixture of vitamin K1 and vitamin K2;
optionally a UV absorber such as beta-carotene,
encapsulated into a starch, such as a modified starch and/or a zeolite.

The modified starch may be any of the cyclodextrins (such as alpha, beta, gamma or modified cyclodextrin), amylose or amylopectin. The modified starch may be sourced from potatoes, wheat, corn or rice or other plant containing starch which is non GMO. The zeolite may be any hydrated aluminosilicate mineral such as analcine, chabazine, heulandite, natrolite, phillipsite and stilbite. By encapsulating the composition into the modified starch and/or a zeolite, the composition is UV stable. In oil the composition has loss of activity. In one embodiment there is binding within the lipophilic portion of the modified starch. In another embodiment there is binding within the pores of the zeolite. Suitably the vitamin K1/K2 and the beta-carotene are present in similar amounts. The starch/zeolite may be present in an amount of 4 times the vitamin. K1/K2 The vitamin K1/K2, beta-carotene and starch/zeolite may be present in amounts of 0.01 wt % up to 99.9 wt % of the composition, for example a range of 1 to 99 wt %, or a range of 1 wt % to 10 wt %, 11 wt % to 20 wt %, 21 wt % to 30 wt %, 31 wt % to 40 wt %, 41 wt % to 50 wt %, 51 wt % to 60 wt %, 61 wt % to 70 wt %, 71 wt % to 80 wt %, 81 wt % to 90 wt % or 91 wt % to 99 wt %, for example 95 wt %, 90 wt %, 80 wt %, 75 wt %, 70 wt %, 60 wt %, 50 wt %, 40 wt %, 30 wt %, 20 wt %, 10 wt %, 5 wt %, 1 wt %, 0.5 wt %, 0.2 wt %, 0.15 wt % or 0.1 wt %.

The composition can include organic or inorganic carriers, excipients and/or diluents. Additives may include emulsifiers/surfactants, thickeners, preservatives, solubilisers, fumed silica or vitamin D3 (cholecalciferol), sweeteners or other suitable additive as desired.

Suitable emulsifiers and thickeners include those having E numbers of E400 to E500.

The emulsifiers may be included in amounts up to 99.9 wt %. For example 1 to 99 wt % or 1 to 10 wt %, 11 to 20 wt %, 21 to 30 wt %, 31 to 40 wt %, 41 to 50 wt %, 51 to 60 wt %, 61 to 70 wt %, 71 to 80 wt %, 81 to 90 wt % or 91 to 99.9 wt % for example a range of 1 to 99 wt %, or a range of 1 wt % to 10 wt %, 11 wt % to 20 wt %, 21 wt % to 30 wt %, 31 wt % to 40 wt %, 41 wt % to 50 wt %, 51 wt % to 60 wt %, 61 wt % to 70 wt %, 71 wt % to 80 wt %, 81 wt % to 90 wt % or 91 wt % to 99 wt %, for example 95 wt %, 90 wt %, 80 wt %, 75 wt %, 70 wt %, 60 wt %, 50 wt %, 40 wt %, 30 wt %, 20 wt %, 10 wt %, 5 wt %, 1 wt %, 0.5 wt %, 0.2 wt %, 0.15 wt % or 0.1 wt %. One suitable emulsifier is polyethoxylated castor oil (PEG35). Another suitable emulsifier is TWEEN 80.

Suitable thickeners include propylene glycol and polyethylene glycol 4000. Thickeners may be included in amounts up to 99.9 wt %. For example 1 to 99 wt % or 1 to 10 wt %, 11 to 20 wt %, 21 to 30 wt %, 31 to 40 wt %, 41 to 50 wt %, 51 to 60 wt %, 61 to 70 wt %, 71 to 80 wt %, 81 to 90 wt % or 91 to 99.9 wt %, for example a range of 1 to 99 wt %, or a range of 1 wt % to 10 wt %, 11 wt % to 20 wt %, 21 wt % to 30 wt %, 31 wt % to 40 wt %, 41 wt % to 50 wt %, 51 wt % to 60 wt %, 61 wt % to 70 wt %, 71 wt % to 80 wt %, 81 wt % to 90 wt % or 91 wt % to 99 wt %, for example 95 wt %, 90 wt %, 80 wt %, 75 wt %, 70 wt %, 60 wt %, 50 wt %, 40 wt %, 30 wt %, 20 wt %, 10 wt %, 5 wt %, 1 wt %, 0.5 wt %, 0.2 wt %, 0.15 wt % or 0.1 wt %.

Suitable preservatives include methyl paraben. Preservatives may be included in amounts up to 2 wt %, for example 1 wt %, 0.5 wt %, 0.2 wt %, 0.1 wt %.

Suitable solubilisers include ethanol. Solubilisers may be included in an amount up to 99.9 wt %. For example 1 to 99 wt % or 1 to 10 wt %, 11 to 20 wt %, 21 to 30 wt %, 31 to 40 wt %, 41 to 50 wt %, 51 to 60 wt %, 61 to 70 wt %, 71 to 80 wt %, 81 to 90 wt % of 91 to 99.9 wt %, for example a range of 1 to 99 wt %, or a range of 1 wt % to 10 wt %, 11 wt % to 20 wt %, 21 wt % to 30 wt %, 31 wt % to 40 wt %, 41 wt % to 50 wt %, 51 wt % to 60 wt %, 61 wt % to 70 wt %, 71 wt % to 80 wt %, 81 wt % to 90 wt % or 91 wt % to 99 wt %, for example 95 wt %, 90 wt %, 80 wt %, 75 wt %, 70 wt %, 60 wt %, 50 wt %, 40 wt %, 30 wt %, 20 wt %, 10 wt %, 5 wt %, 1 wt %, 0.5 wt %, 0.2 wt %, 0.15 wt % or 0.1 wt %.

Suitably the composition is UV stable and has improved bioavailability.

Other additives may include buffers, antioxidants or sweeteners such as dextrose. Other additives may include powderising agents such as Aerosil 200 or icing sugar and viscosifiers such as guar gum or xantham gum.

Excipients can be added to produce a final feed product, by adding vitamins, minerals and grains or grain proteins, such as soya, wheat or corn and grasses such as hay or lucerne.

The composition may be packaged in a syringe, bucket, jar or any other container and may be coloured or contain a UV absorber in the plastic to prevent degradation of the composition by UV during travel or storage.

The invention will now be described by way of example only having regard to the following examples.

The following examples show compositions suitable to make a stable therapeutic dose.

### EXAMPLE 1

Stable Powder Composition Concentrate (A)

5 Kg of vitamin K1 and/or K2 oil was dissolved in 100 L of Ethanol. 20 Kg beta cyclodextrin was heated to 40° C. in 100 L of water. While mixing the cyclodextrin/water solution with high shear (Silverson), the K1/K2 ethanol mixture was slowly added to the cyclodextrin until the vitamin K1 and/or K2 became incorporated into the hydrophobic portion of the clathrate (this can be checked by centrifuging the mixture and testing for Vitamin K in the supematant). The cyclodextrin may be replaced with a zeolite and the K1 and/or K2 becomes incorporated in the pores of the zeolite. To the composition is then added 2 Kg Aerosil 200 by coating the outside of the particles to form a free flowing powder.

### EXAMPLE 2

Stable Powder Composition Concentrate (B)

4.2 Kg of Vitamin K1 and/or K2 oil was added to a water soluble or miscible emulsifier, 4.2 Kg polyoxyl 35 castor oil and mixed. To this paste 87.4 Kg of icing sugar (5% non GMO starch) was added and mixed into a powder with a high shear mixer, then 4.2 Kg of beta carotene (10% WS) was added and thoroughly mixed.

Both stable concentrated powder formulations A and B can be further diluted depending on usage in food or water.

### EXAMPLE 3

Paste Composition using Concentrate A

15 gm Vitamin K1 and/or K2 from concentrate A

11.8 Kg Propylene glycol

7.0 Kg Polyethylene glycol 4000

850 gm Polyethoxylated castor oil (PEG35) or TWEEN 80

15 gm beta carotene WS10%

105 gm Vitamin D3 (500,000 iu/gm) WS

100 gm methyl paraben.

### EXAMPLE 4

Paste Composition using Concentrate A

15 gm Vitamin K1 and/or K2 from concentrate A

850 gm Polyethoxylated castor oil (PEG35) or TWEEN 80

15 gm beta carotene WS10%

105 gm Vitamin D3 (500,000 iu/gm) WS

18.8 Kg Water

100 gm Guar or Xanthan gum

100 gm methyl paraben.

### EXAMPLE 5

Stable Diluted Powder Composition Ready for Use

25 Kg vitamin K1 and/or K2 of concentrate (A)

4.2 Kg Polyoxyl castor oil (PEG35) or TWEEN 80

4.2 Kg beta carotene WS10%

75 Kg Vitamin D3 (500,000 iu/gm) WS

25 Kg Icing sugar (5% corn starch non GMO)

### EXAMPLE 6

Stable Diluted Ready for Use Using Concentrate Powder (B)

1 Kg Vitamin K1 and/or K2 powder concentrate (B)

3 Kg Vitamin D3 (500,000 iu/gm) WS

Made up to 1000 kg with icing sugar or zeolite

### EXAMPLE 7

Stable Diluted Powder in Vitamin Mineral Preblend

10 Kg Vitamin K1 and/or K2 powder (see Example 5 or 6)

100 Kg Vitamin and mineral preblend

### EXAMPLE 8

An experiment was conducted in turkeys. In this experiment all diets used in this study were identical in nutrients, the only difference being that the control diets contained 4 gm/tonne K3 and the experimental diet contained the formulated vitamin KQ at 580 mg/tonne or per 1000 L.

Three groups of 5000 turkeys (15000 turkeys per farm) were used. In the control groups 1 and 3 were fed Vitamin K3 (4 gm/tonne of food). Experimental group 2 was fed Vitamin KQ (580 mg formulated K1/K2/Example 2/tonne of food). 200 turkeys from each group were then weighed at 10 weeks.

It was found that the animals that received vitamin KQ were much more evenly weighed than the control groups. The weight deviation of the experimental group, at an average weight of 6.58 kg, was 100 grams. The control groups had an average weight of 6.97 kg, but with a deviation of up to 1.2 kg. What was evident in the experimental group was that the animals had grown more consistently.

From observation of the animals, it was clearly visible that the turkeys in the experimental group had more vitality than the turkeys in the control group. The turkeys in the control groups showed obvious nervousness and restlessness, particularly when subjected to increasingly stressful situations. The plumage of the turkeys in the control groups was relatively poorly marked, and aggressive pecking was rampant, the animals were dirty and colibacillus infection was evident. The mobility of the turkeys in the control groups was very unstable (their legs were weaker) even though the animals weighed an average of only 6.97 kg.

The turkeys in the experimental group which had been treated with vitamin KQ were much calmer and less stressed and minimal pecking was experienced. The entire barn appeared to be very relaxed. The plumage of the turkeys was considerably better and evenly grown compared to the control groups. The animals were clean and showed even head colouring. Even after weighing, the animals had no problems in their movement.

Although the turkeys in the experimental group were kept in an unfavorable middle barn and the turkeys in the control groups were kept on either side, it was confirmed that there was no suspicion of E. coli infection. Usually it is the middle barn that has the largest infection pressure, as the outside barns on both sides transmit the germs. It was therefore surprising that the turkeys in the experimental group were noticeably more energetic and healthier, their legs were stronger and the animals were more mobile.

### EXAMPLE 9

A second experiment was conducted with turkeys as follows:
Duration of trial: 35 weeks
Age: Supplemented from day 1 onwards
Turkey Amount: 80,000 turkeys.
Experiment group (40,000 turkeys): this group was fed 580 mg KQ per Example 2 per tonne feed
Control group (40,000 turkeys): this group was fed Vitamin K3 (4 gm/tonne) of feed
Dosage: Feed
Fed continually from day 1 onwards.

It was found that the birds in the experimental group gained about 100 grams more weight compared to the control group within the same growing period. The loss rate was lower by about 2% than the control group. The entire trial group was healthier and had more vitality than the control group after just 10 days.

In general, the experimental populations grew evenly, had a very high state of health and were obviously more mobile from the skeletal structure than those in previous rounds.

What was clearly noticeable was that the turkeys in the experiment group had a very healthy intestinal function. Their plumage was very clean and their bedding was dry.

All animals in the experimental group showed a steady growth and steady increase in weight. Also the treating measures with medication were significantly reduced in the experimental group.

### EXAMPLE 10

An experiment was conducted with turkeys.
Duration of trial: 35 weeks
Age: Supplemented from day 7 onwards
Turkey Amount: 10,000
Experimental Group: 6,000 turkeys
Control Group: 4,000 turkeys
Dosage: 580 mg KQ (Example 2) per 1 tonne feed.
Control Group vitamin K3 4 gm/tonne of feed
Fed continually from day 7 onwards.

Again in general, the experimental populations grew evenly, had a very high state of health and were obviously more mobile from the skeletal structure than those in previous rounds. What was clearly noticeable in the turkeys is that the animals had a very healthy intestinal function. The plumage was very clean and the bedding dry.

In various weight checks, there was hardly any difference. All animals in the experimental group showed a steady growth and steady increase in weight as opposed to the turkeys in the control group. Also the treating measures with medication were significantly reduced in the experimental group.

After 10 weeks fattening the animals had a weight gain of approximately 200 grams more than the animals in the control group. The loss rate was already 1.8% lower than in the control group.

### EXAMPLE 11

An experiment was conducted with chickens (broilers). 41,000 chickens were in a control group and were fed 4 gm/tonne vitamin K3. 41,000 thickens were in an experimental group and fed on experimental diet of 580 mg of Vitamin KQ (Example 2) formulated per tonne of food. Results were determined after the chickens were 30 days old.

What was clearly recognizable was the general state of health of the animals on the experimental diet with vitamin KQ. There was minimal pecking and signs of aggression in the treated group as opposed to the control group. Optically the animals carried a clean, dry plumage. At various weighings, a maximum divergence of approximately 30 grams per stable/animal were ascertained. This shows that in the barns, a steady growth and steady increase in weight were recognizable. The bedding was also dry in the treated group as compared to the control group where the bedding was always wet looking and dirty. This means that the treated animals have a healthy, functioning digestive system. The requirement for medication in the treated group was minimal. The vitality of the animals was clearly recognizable.

Except for the minimal treatments, drug application was clearly reduced in the treated group, while the control group had to have prescribed vaccination. This represents an economic benefit, depending on regulatory authorities.

The experience with the broiler's that were given the experimental diet shows that the use of the product is an absolute alternative to other products (drugs).

However, conspicuity still appears with the butchering of the animals. A common occurrence during slaughter is that due to extreme muscle spasms, bones or joints may break. This naturally leads to a devaluation of the slaughter quality. The animals treated with the experimental diet did not exhibit this appearance. Of 41,000 birds slaughtered in the control, there were 3200 birds with broken bones while in the experimental group there were only 1440. There was a 45% less carcass quality reduction on grounds of these bone and joint fractures.

A comparison was made between the following:
Marketable feet A quality in Control group was 81.37%
Marketable feet A quality in experimental group was 87.48%
Skin Infections in control group was 3.65%
Skin infections in experimental group was 1.05%
Dropsy in control group was 0.36%
Dropsy in experimental group was 0.08%.

Marketable animals according an official poultry meat inspector was 95.25% in the control group and 98.25% in the experimental group.

### EXAMPLE 12

Caged layer fatigue is a disease of layer hens involving loss of bone density (osteoporosis) aslaying progresses due to negative calcium balance. Treatment with KQ (Example 2) reversed the process. (Treatment with menadione failed to prevent the disease.)

TABLE 1
X-rays of bone density as measured as RBAE (Radiographic bone
aluminium equivalents) mm provided the following results:

| Vitamin K | Day 27 | Day 35 | Change % |
|---|---|---|---|
| 250 ug K3 menadione/bird/day | 3.85 | 3.64 | −5.4 |
| 250 ug Vitamin KQ/bird/day | 3.88 | 4.04 | +3.8 |

It was noted that menadione (K3) deceased bone growth whilst Vitamin KQ increased bone strength and minimized bone breakage. Vitamin KQ boosted the carboxylation of osteocalcin and decreased the concentration of serum under-carboxylated osteocalcin enhancing hydroxyapatite binding capacity of serum osteocalcin and improving bone quality.

### EXAMPLE 13

An experiment was conducted with laying birds. 8,000 birds were used (there was no control group). Age of the birds were: ca. 13 months. Dosage: 1 L vitamin KQ (Example 2) mixed in 1000 L (24 hour period) every 3 days.

Prior to the experiment, laying percentage was a consistent 45% usable eggs (plus 10 shell-less eggs). This corresponds to a weekly average of 3,600 eggs per day (45%).

On beginning the experiment, in 8 days the percentage went up to 78% and stayed constant for the following 14 days of the trial. This corresponds to a weekly average increase of up to 6,240 eggs per day. There were no shell-less eggs.

An improvement in egg quality was noticed (size and shape) and all the animals showed more vitality. They were calmer, cleaner and their bedding was dryer. They showed less agitation and less gastrointestinal problems.

### EXAMPLE 14

An experiment was conducted with old laying birds aged 21 weeks. 20,000 birds were given 580 mg formulated vitamin KQ/tonne of food (Example 2), instead of the control diet of 4 gm/tonne Vitamin K3.

Layers went from 14,800 eggs per day in the control group to 18,200 in the treated group and the animals were cleaner with less gastrointestinal problems in the treated group. The birds in the treated group also produced a stronger egg due to an increase in calcium in the shell. There was also 1-2% less breakage in the treated group. See Table 2:

TABLE 2
% eggs layed as the layers age comparing control versus treatment

| Age of layers | Control | Treated |
|---|---|---|
| 2 weeks | 92% | 96% |
| 21 weeks | 92% | 96% |
| 60 weeks | 74% | 91% |
| 90 weeks | 54% | 81% |

### EXAMPLE 15

An experiment was conducted with laying birds and broilers:
A) Chickens hatched from the breeder layer eggs (fed 580 mg/tonne of formulated vitamin KQ) contained 50-100 ug Vitamin K1 per egg from day 1 (Example 2);
B) Chickens hatched from the breeder layer eggs were fed 4 gm/tonne vitamin K3 from day 1 contained 4-10 ug vitamin K1 per egg from day 1.

1000 chickens from each group were slaughtered at day 35 and X-rayed. See Table 3.

TABLE 3
X-rays of bone density as measured as RBAE (Radiographic
bone aluminium equivalents) mm:

| Vitamin K | Day 35 | Bone Ash |
|---|---|---|
| 4 gm vitamin | 3.64 | 35% |
| K3/tonne feed | | |
| 580 mg vitamin | 6.04 | 42% |
| KQ/tonne of feed | | |

It was noted that chickens on menadione K3 had a bone density 60% of that of Vitamin KQ and bone ash 83%.

It was also noted that when the egg was hatched from an egg which contained vitamin KQ by feeding the breeder formulated vitamin K, the results in bone strength were superior than chickens fed from birth. Compare Table 3 against Table 1 week 35 (X-ray results 4.04 compare to 6.04).

### EXAMPLE 16

An experiment was conducted with ducks. In the duck's rearing/fattening a similar experience was found as with chickens and turkeys with respect to observations of weight, welfare and behaviour.

Duration of trial: 16 weeks
Age: Supplemented from day 7 onwards
Amount: 7,500 birds
Experimental Group: 3,500 birds
Control Group: 4,000 birds
Dosage: Treatment 580 mg KQ per 1000 L water (Example 2). Control 4 gm vitamin K3 per 1000 L water
Dosage given for 24 hours 3× per week.

The ducks beaks were not capped. All birds were of the same average weight in the treated group compared to larger variations in the control group. All the birds appeared calmer and healthier showing more vitality than the control group. Their plumage was much cleaner compared to the control group and the bedding was considerably cleaner and drier than the control group.

Up until the 10th week, there was no feather picking at all in the treated group. In the 11th week there were occasional cases of feather picking. Compared to the control group in which there was extensive feather picking. Supplementation was doubled (1160 mg Vit KQ/1000 L water) and given for 48 hrs. Feather picking stopped completely.

### EXAMPLE 17

An experiment was conducted with pigs. Quinaquanone was used with breeding sows. The aim was to reduce the birth losses by application of the composition. It was thought that stillbirths could be prevented by strengthening the immune system as well as improving the vitality of the piglets. The experimental group received 580 mg of formulated KQ (Example 2) per 1000 litres of drinking water (compared to the control which was 4 gm vitamin K3 per 1000 L drinking water). Permanent administration showed an improvement in the vitality of the breeding sows after approximately 10 days compared to the control.

For this trial, a barn with 20 breeding sows was selected. Typically, the birth rate was about 33 piglets born. On average, this accounts for three stillborn piglets and three piglets being crushed due to lack of vitality of the sows.

A test was conducted to determine if the supply of vitamin KQ has an influence on stillbirths. Vitamin KQ was used approximately 19 days prior to birth. During this period, the sows were kept in farrowing crates so that the sows were supplied with optimal vitamin KQ. The supply of the sow's vitamin K1 rich milk improved the immune system of the piglets significantly. The vitality of the piglets was also improved compared to the control.

In the natal phase influence on the stillbirths was approximately 1.5%. This was attributed by veterinarians to the improved vitality of the breeding sows. The live-born piglets showed a faster mobility of the musculoskeletal system compared to the control group. It was more decisively recognizable with the 25 kg piglets that they did not suffer from joint or bone deformations. This observation was also made on 3 different farms.

As mentioned above with respect to poultry, the arrangements of the microbes in the intestinal tract are jointly responsible for the immune system, so that it can be assumed that bacteria and illness caused by Clostridium and Streptococcus was restrained by the immune system. This is a decisive product advantage in pork that can be used.

The foregoing description of preferred embodiments and best mode of the invention known to the applicant at the time of filing the application have been presented for the purposes of illustration and description. It is not intended to be exhaustive or to limit the invention to the precise form disclosed. Many modifications and variations are possible in the light of the above teaching. The embodiments were chosen and described in order to best explain the principles of the invention and its practical application to thereby enable others skilled in the art to best utilize the invention in various embodiments and with various modifications as are suited to the particular use contemplated. It is intended that the scope of the invention be defined by the claims appended hereto.

1-6. (canceled)

7. A method of improving the wellbeing, as herein defined, of a pig, turkey, duck or chicken comprising administering to an animal an effective amount of UV stable, water soluble composition containing:
vitamin K1, vitamin K2 or a mixture of vitamin K1 and vitamin K2, together with a UV absorber and physiologically acceptable carrier, excipient and/or diluent;
wherein the composition includes an emulsifier and/or wherein the vitamin K1, vitamin K2 or mixtures thereof is encapsulated into a modified starch and/or zeolite.

8. The method according to claim 7 wherein the composition is administered orally.

9. The method according to claim 7 wherein the composition contains vitamin D.

10. The method according to claim 7 wherein the composition comprises: vitamin K1, vitamin K2 or a mixture of vitamin K1 and vitamin K2; beta-carotene; and an emulsifier.

11. The method according to claim 7 wherein the composition is a stable powder composition comprising:
vitamin K1, vitamin K2 or a mixture of vitamin K1 and vitamin K2; and beta-carotene; an emulsifier and/or thickening agent encapsulated into a modified starch and/or zeolite.

12. The method according to claim 7 wherein the composition is a stable powder composition comprising;
vitamin K1, vitamin K2 or a mixture of vitamin K1 and vitamin K2;
beta-carotene;
Vitamin D;
an emulsifier;
encapsulated into a modified starch and/or zeolite.
